(19) United States
(12) Patent Application Publication
(10) Pub. No.: US 2017/0090475 A1
(43) Pub. Date: Mar. 30, 2017

(54) DRIVER MONITORING METHOD AND APPARATUS USING WEARABLE DEVICE

(71) Applicant: SAMSUNG ELECTRONICS CO., LTD., Suwon-si (KR)

(72) Inventors: Changmok CHOI, Yongin-si (KR); Byunghoon KO, Hwaseong-si (KR); TakHyung LEE, Suwon-si (KR); Sang Joon KIM, Hwaseong-si (KR)

(73) Assignee: SAMSUNG ELECTRONICS CO., LTD., Suwon-si (KR)

(21) Appl. No.: 15/188,074

(22) Filed: Jun. 21, 2016

(30) Foreign Application Priority Data
Sep. 24, 2015 (KR) 10-2015-0135573

(51) Int. Cl. G05D 1/00; A61B 5/0205; A61B 5/00; A61B 5/0402; A61B 5/18; A61B 5/0488; A61B 5/04; A61B 5/145; A61B 5/1455; B60W 40/08; A61B 5/0476

(52) U.S. Cl. CPC G05D 1/0061; B60W 40/08; A61B 5/0205; A61B 5/0059; A61B 5/0402; A61B 5/0476; A61B 5/0488; A61B 5/04001; A61B 5/14532; A61B 5/14552; A61B 5/18; B60W 2040/0818; A61B 5/021

(57) ABSTRACT

A driver monitoring method and apparatus is disclosed. The apparatus may control an activation of monitoring devices provided in a vehicle based on detecting a movement of a driver, which is received from a wearable device worn on the driver, and to determine whether the driver is in a normal driving state using information collected from the activated monitoring devices. A driver monitoring method may include determining a movement of a driver based on a wearable device, controlling an activation of monitoring devices provided in a vehicle based on the determining of the movement of the driver, collecting information from the monitoring devices, in response to the monitoring device being activated, and determining whether the driver is in a normal driving state in which the driver is able to drive the vehicle normally using the collected information.

### CROSS-REFERENCE TO RELATED APPLICATION(S)

This application claims the benefit under 35 USC §119(a) of Korean Patent Application No. 10-2015-0135573, filed on Sep. 24, 2015, in the Korean Intellectual Property Office, the entire disclosure of which is incorporated herein by reference for all purposes.

### BACKGROUND

1. Field

The following description relates to a driver monitoring method and apparatus using a wearable device.

2. Description of Related Art

As traffic-related death rate increase every year, approximately one fourth of traffic deaths are attributed to careless drivers or drowsy driving. Elderly drivers are also increasing due to an aging population. Traffic accidents are also caused by a health issue, for example, a stroke while driving. When a driver is drowsy or the driver encounters an abnormal health state, discovering such cases early and providing a warning may prevent an accident from happening.

Various sensors may be used to detect a state of a driver while driving, and most of such sensors may be related to high-power devices consuming a great amount of electric power.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, there is provided a driver monitoring method including determining a movement of a driver based on a wearable device, controlling an activation of monitoring devices provided in a vehicle based on the determining of the movement of the driver, collecting information from the monitoring devices, in response to the monitoring device being activated, and determining whether the driver is able to drive the vehicle using the collected information.

The wearable device may include at least one of a biosignal sensor, a motion sensor, an accelerometer, an ultrasonic sensor, a gyro sensor, or an image sensor, and a movement of the driver may be detected by at least one of the motion sensor, the ultrasonic sensor, the accelerometer, the gyrosensor, or the image sensor.

In response to a movement of the driver not being detected for a period of time, the wearable device may be further configured to generate a message indicating no movement of the driver.

In response to the message indicating no movement of the driver being generated, the wearable device may be further configured to measure a biosignal of the driver and may determine whether an abnormal health condition occurs in the driver.

In response to determining an abnormal health condition, the wearable device may be further configured to generate a message indicating an occurrence of the abnormal health condition.

In response to the message indicating no movement of the driver, the method may include measuring a biosignal of the driver using the wearable device, and determining whether an abnormal health condition occurs in the driver based on the measured biosignal.

The determining of the movement of the driver may include receiving the message indicating no movement of the driver.

The controlling of the activation of the monitoring devices may include setting an operation mode of the monitoring devices to be an active mode in response to receiving the message indicating no movement of the driver.

The controlling of the activation of the monitoring devices may include setting an operation mode of the monitoring devices to be a sleep mode, in response to determining a movement of the driver.

The determining of whether the driver is in the normal driving state may include determining whether the driver is able to drive the vehicle using information collected from the monitoring devices and information collected from the wearable device.

The determining of whether the driver is able to drive the vehicle may include determining whether the driver is able to drive the vehicle, in response to a switch of a driving mode of the vehicle from an autonomous driving mode to a manual driving mode.

The determining of whether the driver is is able to drive the vehicle may include estimating whether the driver is drowsy or whether an abnormal health condition occurs in the driver using the collected information, and determining whether the driver is able to drive the vehicle based on the estimating.

The monitoring devices may include at least one of a biosignal sensor configured to sense a biosignal of the driver, a motion sensor configured to sense a movement of the driver, a tactile sensor configured to sense a contact with the driver, or a camera configured to capture an image of at least a portion of the driver's body.

In response to a determination that the driver is not is able to drive the vehicle, the method may include at least one of wirelessly transmitting a warning signal to at least one of a control server and another vehicle traveling around the vehicle, or transmitting a biosignal of the driver and information about the vehicle to the control server.

In response to a determination that the driver is not able to drive the vehicle, the method may include setting a driving mode of the vehicle to an autonomous driving mode.

In another general aspect, there is provided a driver monitoring apparatus, including a receiver configured to receive information, detected from a wearable device worn by a driver, concerning a movement of the driver, and a processor configured to control an activation of monitoring devices provided in a vehicle, in response to the information, and to determine whether the driver is able to drive the vehicle using information collected from the monitoring devices activated, in response to the information indicating no movement of the driver, and wherein the monitoring devices are configured to monitor a movement of the driver.

In response to a movement of the driver not being detected for a period of time, the wearable device may be further configured to generate a message indicating no movement of the driver, to measure a biosignal of the driver, and to determine, based on the measured biosignal, an occurrence of abnormal health condition in the driver.

The processor may be further configured to set an operation mode of the monitoring devices to be an active mode in response to the information indicating no movement of the driver, and to set the operation mode of the monitoring devices to be a sleep mode in response to the information indicating movement of the driver.

The processor may be further configured to estimate whether the driver is drowsy or whether an abnormal health condition occurs in the driver using the collected information, and to determine whether the driver is in the normal driving state based on the estimating.

In another general aspect, there is provided a driver monitoring method including determining a movement of a driver based on at least one of a wearable device or a sensor provided in a vehicle, controlling an activation of monitoring devices provided in a vehicle based on the movement of the driver, determining whether the driver is able to drive the vehicle based on information collected from the monitoring devices, and switching a driving mode of the vehicle to an autonomous driving mode, in response to determining that the driver is unable to drive the vehicle.

The method may include transmitting a warning signal to another vehicle traveling around the vehicle, in response to determining that the driver is unable to drive the vehicle.

The method may include transmitting information about the vehicle and a warning signal to a control server, in response to determining that the driver is unable to drive the vehicle.

Other features and aspects will be apparent from the following detailed description, the drawings, and the claims.

### BRIEF DESCRIPTION OF THE DRAWINGS

FIG. 1 is a diagram illustrating an example of a driver monitoring system.

FIG. 2 is a diagram illustrating an example of a driver monitoring method.

FIG. 3 is a diagram illustrating another example of a driver monitoring method.

FIG. 4 is a diagram illustrating still another example of a driver monitoring method.

FIG. 5 is a diagram illustrating an example of a driver monitoring method performed by a driver monitoring system.

FIG. 6 is a diagram illustrating an example of a driver monitoring apparatus.

Throughout the drawings and the detailed description, unless otherwise described or provided, the same drawing reference numerals will be understood to refer to the same elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent to one of ordinary skill in the art. The sequences of operations described herein are merely examples, and are not limited to those set forth herein, but may be changed as will be apparent to one of ordinary skill in the art, with the exception of operations necessarily occurring in a certain order. Also, descriptions of functions and constructions that are well known to one of ordinary skill in the art may be omitted for increased clarity and conciseness.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

Terms such as first, second, A, B, (a), (b), and the like may be used herein to describe components. Each of these terminologies is not used to define an essence, order or sequence of a corresponding component but used merely to distinguish the corresponding component from other component(s). For example, a first component may be referred to a second component, and similarly the second component may also be referred to as the first component.

It should be noted that if it is described in the specification that one component is “connected,” “coupled,” or “joined” to another component, a third component may be “connected,” “coupled,” and “joined” between the first and second components, although the first component may be directly connected, coupled or joined to the second component. In addition, it should be noted that if it is described in the specification that one component is “directly connected” or “directly joined” to another component, a third component may not be present therebetween. Likewise, expressions, for example, “between” and “immediately between” and “adjacent to” and “immediately adjacent to” may also be construed as described in the foregoing.

The terminology used herein is for the purpose of describing particular embodiments only and is not intended to be limiting. As used herein, the singular forms “a,” “an,” and “the,” are intended to include the plural forms as well, unless the context clearly indicates otherwise. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Examples to be described hereinafter may be used to monitor a driver. The examples may be provided in various devices, such as, for example, a tablet computer, a smartphone, a cellular phone, a smart home appliance, an intelligent vehicle, or a wearable device (such as, for example, a ring, a watch, a pair of glasses, glasses-type device, a bracelet, an ankle bracket, a belt, a necklace, an earring, a headband, a helmet, or a device embedded in the cloths). For example, when an abnormal health state occurs in a user or when the user is drowsy, the devices may determine such an event and provide a suitable measure based on a heart rate of the user, which is measured by the device. The device may be provided in an intelligent vehicle in which a driving mode is switchable from an autonomous driving mode to a manual driving mode or vice versa.

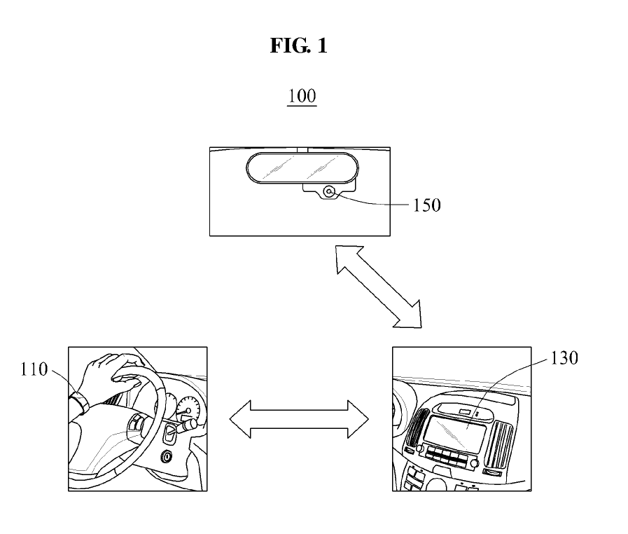

FIG. 1 is a diagram illustrating an example of a driver monitoring system 100. Referring to FIG. 1, the driver monitoring system 100 includes a wearable device 110 and a driver monitoring apparatus 130. The driver monitoring system 100 may include a camera 150. The camera 150 may be included in the driver monitoring apparatus 130 or provided as a separate device.

The wearable device 110 worn by a driver and may detect a biosignal of the driver and a movement of the driver through various sensors. The wearable device 110 may be worn, for example, as a watch, bracelet, chest strap, chest patch, patch, glasses, headband, earring, ankle bracket, belt, earring, headband, helmet, necklace or a device embedded in the cloths.

The wearable device 110 may include a biosignal sensor, a motion sensor, an accelerometer, and a gyrosensor. The biosignal sensor may be, for example, a photoplethysmogram (PPG) sensor. The biosignal sensor may measure a biosignal, such as, for example, an electrocardiogram (ECG), an electromyogram (EMG), a PPG, an electroneurogram (ENG), an electroencephalogram (EEG), a heart rate, a blood pressure, a blood sugar, an oxygen saturation level (SpO₂), a respiration, a pulse, a galvanic skin response (GSR), or an impedance of the driver. The motion sensor, the accelerometer, and the gyrosensor may detect or measure a physical movement of the driver. In an example, one or more of the sensors describe above may be provided in the driver monitoring apparatus 130 or may be provided as separate devices in the vehicle.

While driving a vehicle, the driver may move portions of the body, such as, for example, an arm, in a normal state, and not in a drowsy state or a shocked state. Thus, the wearable device 110 may monitor a movement of an arm or other portions of the driver's body using the motion sensor. Due to an extremely low amount of power consumption of the motion sensor, power consumed by the activation of the motion sensor may not be significant.

When a movement of the arm or other portions of the body of the driver is detected by the motion sensor, the wearable device 110 may determine that the driver is in an awake state in which the driver is awake. In response to a determination that the driver is in the awake state, the wearable device 110 may transmit no signal to the driver monitoring apparatus 130.

When a movement of the arm or other portions of the body of the driver is not detected by the motion sensor for a period of time, the wearable device 110 may determine that the driver is drowsy or that a health issue, hereinafter also referred to as an abnormal health condition, has occurred.

When a movement of the driver is not detected by the motion sensor for a period of time, the wearable device 110 may generate a message and transmit the generated message to the driver monitoring apparatus 130. The wearable device 110 may verity whether there is an abnormality in a health state of the driver by activating the biosignal sensor included in the wearable device 110.

In response to a movement of the driver being detected, the wearable device 110 may maintain a sleep mode of the biosignal sensor. In response to a movement of the driver not being detected, the wearable device 110 may activate the biosignal sensor to measure a biosignal of the driver.

When the wearable device 110 continuously measures a biosignal of the driver, the battery may be discharged due to a large consumption of power. In the present example, when a movement of the driver is not detected, the wearable device 110 may activate the biosignal sensor, and thus, may reduce an amount of battery consumption of the wearable device 110.

In response to a determination that an abnormal health condition occurs in the driver, the wearable device 110 may generate a message indicating the occurrence of the abnormal health condition in the driver and may transmit the generated message to the driver monitoring apparatus 130. In another example, the wearable device 110 may transmit a signal indicating an emergency to a hospital or a police station, or transmit the signal indicating the emergency to a hospital or a police station through a smartphone of the driver interworking with the wearable device 110.

In response to no movement of the driver being detected and no abnormal health condition being determined by the wearable device 110, the driver monitoring apparatus 130 may determine that the driver is in a drowsy state. The driver monitoring apparatus 130 may wake up the driver, or wirelessly transmit a warning signal to other vehicles traveling around the vehicle of the drowsy driver.

The driver monitoring apparatus 130 may monitor the driver by controlling an activation or inactivation of monitoring devices provided in the vehicle based on detecting a movement of the driver by the wearable device 110.

The driver monitoring apparatus 130 may reduce an amount of power consumed by the driver monitoring apparatus 130 by setting an operation mode of the monitoring devices to be a sleep mode and maintaining the sleep mode until the message indicating no movement of the driver for a period of time is received from the wearable device 110. In response to a receipt of the message indicating no movement of the driver from the wearable device 110, the driver monitoring apparatus 130 may set the operation mode of the monitoring devices to be an active mode and activate the monitoring devices.

The monitoring devices may include devices, such as, for example, a biosignal sensor configured to sense a biosignal of the driver, a motion sensor configured to sense a movement of the driver, an accelerometer, a gyro sensor, a tactile sensor configured to sense a contact with the driver, an ultrasonic sensor, and a camera or an image sensor configured to capture an image of the driver.

The driver monitoring apparatus 130 may monitor the driver using the monitoring devices or additional external sensors.

The driver monitoring apparatus 130 may determine whether the driver is in a normal driving state in which the driver is able to drive the vehicle normally using information collected from the monitoring devices and information collected from the wearable device 110. Here, the normal driving state indicates a state in which the driver is able to drive the vehicle normally without being drowsy while driving the vehicle, without a physical abnormality occurring due to the influence of alcohol or drugs, or without a physical abnormality such as a shock.

When a drowsiness or physical abnormality is detected, the driver monitoring apparatus 130 may generate a warning signal or an alarm to wake up the driver. The driver monitoring apparatus 130 may also transmit a warning message to warn vehicles traveling around the vehicle of a potential danger that may be caused by the drowsiness or physical abnormality of the driver and to warn the vehicles to be more careful about safety. In another example, the driver monitoring apparatus 130 may transmit a help-seeking message to an emergency service and a police station through a communication network.

When an abnormal health condition does not occur in the driver and the driver is not drowsy, despite the receipt of the message indicating no movement of the driver, the driver monitoring apparatus 130 may determine that the driver is simply in a still state, for example, the driver is waiting at a traffic signal.

For an intelligent vehicle where a driving mode is switchable between an autonomous driving mode and a manual driving mode, the driver monitoring apparatus 130 may verify whether the driver is in the normal driving state. In response to a determination that the driver is drowsy in the manual driving mode, the driver monitoring apparatus 130 may switch the driving mode of the vehicle to the autonomous driving mode.

The driver monitoring apparatus 130 may be included in an in-vehicle infotainment in which a navigation, an audio, and a video function, and the Internet are combined. In another example, the driver monitoring apparatus 130 may be included in a smartphone or a driver monitoring system, which is an aftermarket product.

The driver monitoring apparatus 130 may be connected to the wearable device 110 through wired or wireless communication. The driver monitoring apparatus 130 may directly communicate with the wearable device 110, or may be connected to the wearable device 110 through a control server.

The camera 150 may be activated by a control signal of the driver monitoring apparatus 130, and may capture an image of the driver. For example, the camera 150 may capture a driving behavior or a face of the driver, or track a gaze of an eye of the driver.

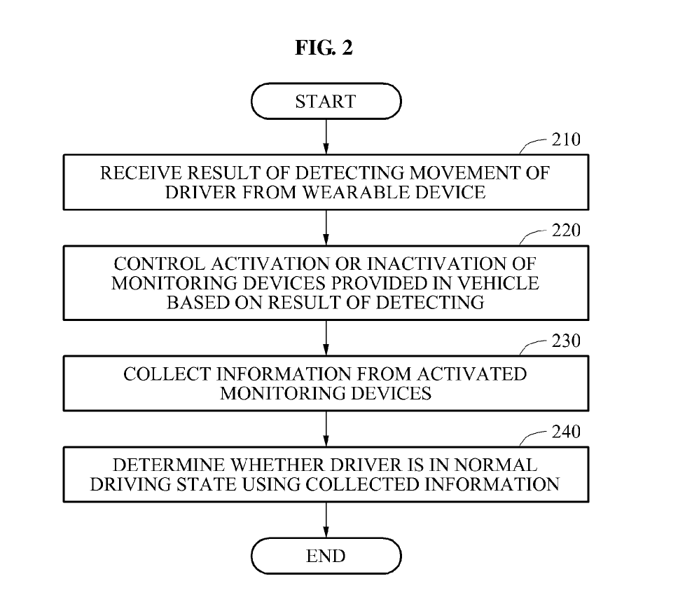

FIG. 2 is a diagram illustrating an example of a driver monitoring method. The operations in FIG. 2 may be performed in the sequence and manner as shown, although the order of some operations may be changed or some of the operations omitted without departing from the spirit and scope of the illustrative examples described. Many of the operations shown in FIG. 2 may be performed in parallel or concurrently. The above descriptions of FIG. 1 is also applicable to FIG. 2, and is incorporated herein by reference. Thus, the above description may not be repeated here.

Referring to FIG. 2, in 210, a driver monitoring apparatus receives a result of detecting a movement of a driver from a wearable device worn by the user. As described above, the wearable device may include, for example, a biosignal sensor, a motion sensor, an accelerometer, and a gyrosensor. A movement of the driver may be detected by at least one of the motion sensor, the accelerometer, and the gyrosensor. A movement of the driver may include a movement of a physical portion of the driver on which the wearable device is worn and a movement of other physical portions of the driver.

In response to a movement of the driver not being detected for a period of time, the wearable device may generate a message indicating no movement of the driver has been detected and may transmit the generated message to the driver monitoring apparatus. In an example, the result of the detecting of a movement of the driver may be, for example, the message indicating no movement of the driver.

In 220, the driver monitoring apparatus controls an activation or inactivation of monitoring devices provided in a vehicle of the driver. The driver monitoring apparatus may set an operation mode of the monitoring devices to be an active or a sleep mode based on whether or not a message indicating no movement of the driver is received.

According to examples, the driver monitoring apparatus may activate all the monitoring devices, or activate a portion of the monitoring devices as desired.

In 230, the driver monitoring apparatus collects information from the activated monitoring devices. The monitoring devices may be installed inside the vehicle or on other locations outside the vehicle. The monitoring devices may be installed in locations, such as, for example, a steering wheel, a rear-view mirror, and a driver seat of the vehicle. The monitoring devices may include, for example, a biosignal sensor configured to sense a biosignal of the driver, an accelerometer, a gyro sensor, a motion sensor configured to sense a movement of the driver, a tactile sensor configured to sense a contact with the driver, an ultrasonic sensor, and a camera or an image sensor configured to capture an image of the driver.

The driver monitoring apparatus may collect information about the driver, such as, for example, a heart rate, a respiration, or a temperature of the driver, from the biosignal sensor and collect a result of detecting a movement of the driver from the motion sensor, the accelerometer, and the gyrosensor. The driver monitoring apparatus may collect information about the driver, such as, for example, a contact or a pressure, which indicates whether the driver holds the steering wheel, from the tactile sensor. The driver monitoring apparatus may collect, from the camera, information about the driver, such as, for example, an image obtained by tracking and capturing a driving behavior, a face, or a gaze of an eye of the driver.

In 240, the driver monitoring apparatus determines whether the driver is in a normal driving state in which the driver is able to drive the vehicle normally using the information collected from the monitoring devices. For example, when the heart rate, the respiration, or the temperature is 5% higher or lower than respective normal values, the driver monitoring apparatus may determine or estimate that an abnormal health condition has occurred for the driver. In another example, when the contact with the driver or the pressure against the steering wheel, which is detected by the tactile sensor, is lower than an average value, the eye of the driver captured through the camera is closed or the gaze moves unstably in a short period of time without being fixed, or the face of the driver nods in a direction, the driver monitoring apparatus may determine or estimate that the driver is drowsy. For example, the driver monitoring apparatus may determine a direction of the gaze of the driver and whether the eye of the driver is closed in the captured image using a facial recognition algorithm or an eye tracking algorithm.

The driver monitoring apparatus may determine whether the driver is in the normal driving state using the information collected from the monitoring devices and the information collected from the wearable device.

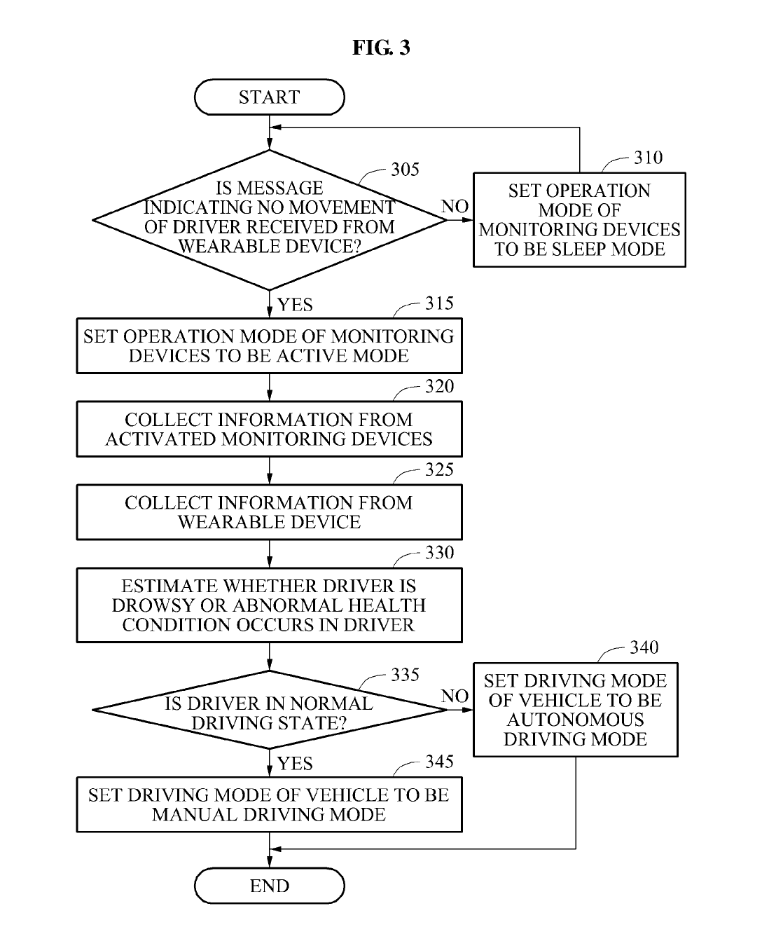

FIG. 3 is a diagram illustrating another example of a driver monitoring method. The operations in FIG. 3 may be performed in the sequence and manner as shown, although the order of some operations may be changed or some of the operations omitted without departing from the spirit and scope of the illustrative examples described. Many of the operations shown in FIG. 3 may be performed in parallel or concurrently. The above descriptions of FIGS. 1-2 is also applicable to FIG. 3, and is incorporated herein by reference. Thus, the above description may not be repeated here.

Referring to FIG. 3, in 305, a driver monitoring apparatus determines whether a message indicating no movement of a driver is received from a wearable device. In 310, in response to not receiving a message indicating no movement of the driver, the driver monitoring apparatus sets an operation mode of monitoring devices to be a sleep mode. The driver monitoring apparatus may return to operation 305 and wait for the message indicating no movement of the driver to be received.

In 315, in response to receiving a message indicating no movement of the driver, the driver monitoring apparatus sets the operation mode of the monitoring devices to be an active mode. In 320, the driver monitoring apparatus collects information from the activated monitoring devices. In 325, the driver monitoring apparatus collects information from the wearable device.

In 330, using the information collected from the monitoring devices and the wearable device, the driver monitoring apparatus estimates whether the driver is drowsy or an abnormal health condition occurs in the driver. The information collected from the monitoring devices and the wearable device may be a result of detection performed by various sensors included in the monitoring devices and the wearable device.

For example, the driver monitoring apparatus may estimate a degree of fatigue of the driver or estimate whether the driver is drowsy by measuring a percentage of eye closure (PERCLOS) and an eye saccade based on information collected from a camera or an image sensor. The eye saccade may be construed as a fast and simultaneous movement of both eyes in a same direction. For example, the driver monitoring apparatus may estimate that the abnormal health condition occurs in the driver in response to a heart rate, a respiration, or a temperature of the driver being 5% higher or lower than respective normal values.

In 335, the driver monitoring apparatus determines whether the driver is in a normal driving state. For example, the driver monitoring apparatus may determine whether the driver is in the normal driving state when switching a driving mode of a vehicle of the driver from an autonomous driving mode to a manual driving mode.

In 340, in response to a determination that the driver is not in the normal driving state, the driver monitoring apparatus sets the driving mode of the vehicle to be the autonomous driving mode. If needed, the driver monitoring apparatus may force the driving mode of the vehicle to be the autonomous driving mode.

In 345, in response to a determination that the driver is in the normal driving state, the driver monitoring apparatus sets the driving mode of the vehicle to be the manual driving mode.

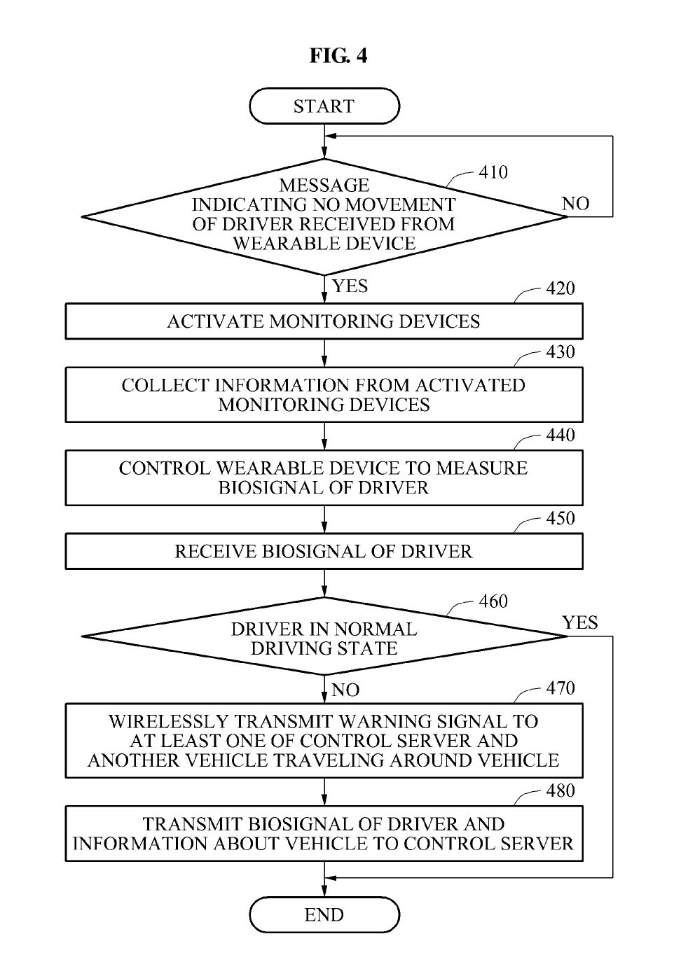

FIG. 4 is a flowchart illustrating still another example of a driver monitoring method in accordance with an embodiment. The operations in FIG. 4 may be performed in the sequence and manner as shown, although the order of some operations may be changed or some of the operations omitted without departing from the spirit and scope of the illustrative examples described. Many of the operations shown in FIG. 4 may be performed in parallel or concurrently. The above descriptions of FIG. 1-3 is also applicable to FIG. 4, and is incorporated herein by reference. Thus, the above description may not be repeated here.

Referring to FIG. 4, in 410, a driver monitoring apparatus determines whether a message indicating no movement of a driver is received from a wearable device. When a message indicating no movement of the driver is not received, the driver monitoring apparatus may wait for the message indicating no movement of the driver to be received.

In 420, in response to a determination that the message indicating no movement of the driver is received, the driver monitoring apparatus activates monitoring devices. In 430, the driver monitoring apparatus collects information from the activated monitoring devices.

In 440, in response to the determination that the message indicating no movement of the driver is received, the driver monitoring apparatus controls the wearable device to measure a biosignal of the driver. In 450, the driver monitoring apparatus receives the biosignal of the driver from the wearable device. According to examples, 440 may be performed concurrently with 420, or performed between 420 and 430, or performed prior to 420.

In 460, the driver monitoring apparatus determines whether the driver is in a normal driving state in which the driver is able to drive a vehicle normally based on the biosignal of the driver, which is received in 450 and the information collected from the monitoring devices in 430.

In 470, in response to a determination that the driver is not in the normal driving state, the driver monitoring apparatus transmits a warning signal to at least one of a control server and another vehicle traveling around or near the vehicle. In an example, the transmitting of the warning signal may be done wirelessly.

In 480, the driver monitoring apparatus transmits the biosignal of the driver and information about the vehicle to the control server.

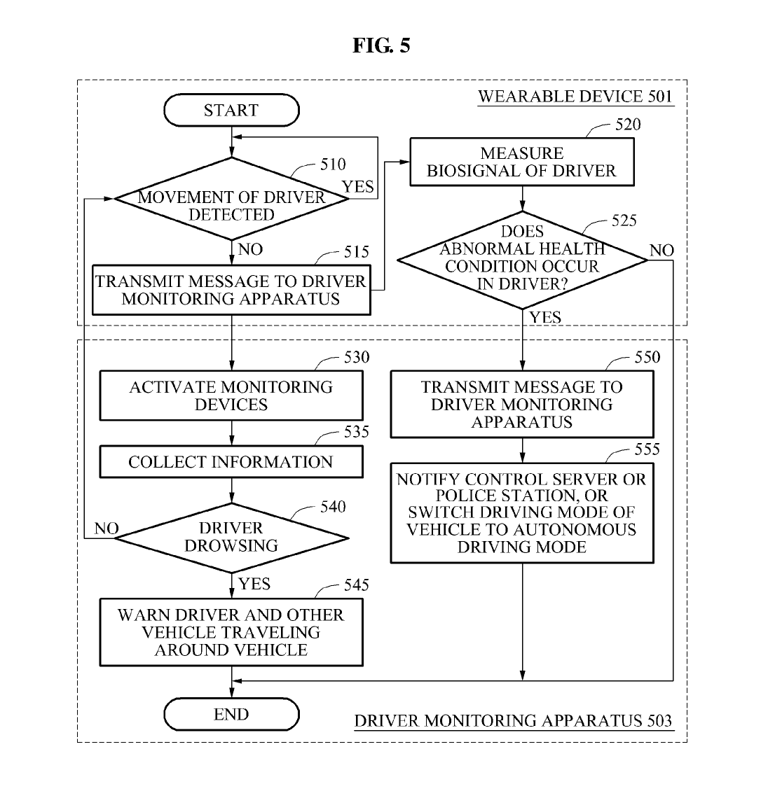

FIG. 5 is a diagram illustrating an example of a driver monitoring method performed by a driver monitoring system. The operations in FIG. 5 may be performed in the sequence and manner as shown, although the order of some operations may be changed or some of the operations omitted without departing from the spirit and scope of the illustrative examples described. Many of the operations shown in FIG. 5 may be performed in parallel or concurrently. The above descriptions of FIG. 1-4 is also applicable to FIG. 5, and is incorporated herein by reference. Thus, the above description may not be repeated here.

Referring to FIG. 5, the driver monitoring method is performed with a wearable device 501 and a driver monitoring apparatus 503, both of which may be included in the driver monitoring system.

In 510, the wearable device 501 determines a presence or absence of a movement of a driver based on a movement of the driver determined by a motion sensor. In response to a determination of the presence of the movement of the driver, the wearable device 501 is on standby.

In 515, in response to a determination of the absence of the movement of the driver, the wearable device 501 transmits a message to the driver monitoring apparatus 503. For example, the message indicates the absence of the movement of the driver.

In 520, the wearable device 501 measures a biosignal of the driver after transmitting the message to the driver monitoring apparatus 503.

In 525, the wearable device 501 determines whether an abnormal health condition occurs in the driver based on a result of the measuring the biosignal. In 550, in response to a determination that the abnormal health condition occurs in the driver, the wearable device 501 transmits a message indicating occurrence of the abnormal health condition to the driver monitoring apparatus 503.

In 555, the driver monitoring apparatus 503 receiving the message indicating the occurrence of the abnormal health condition provides a notification of such information to a control server or a police station. In another example, in 555, the driver monitoring apparatus 503 switches a driving mode of a vehicle of the driver to an autonomous driving mode. If needed, the driver monitoring apparatus may force the driving mode of the vehicle to be the autonomous driving mode.

In 530, the driver monitoring apparatus 503 receives the message indicating the absence of the movement of the driver from the wearable device 501, and activates monitoring devices provided in the vehicle or outside the vehicle.

In 535, the driver monitoring apparatus 503 collects information from the activated monitoring devices. In 540, the driver monitoring apparatus 503 determines whether the driver is drowsy using the collected information.

In 545, in response to a determination that the driver is drowsy, the driver monitoring apparatus 503 warns the driver or another vehicle traveling around or near the vehicle through an alarm or a warning message.

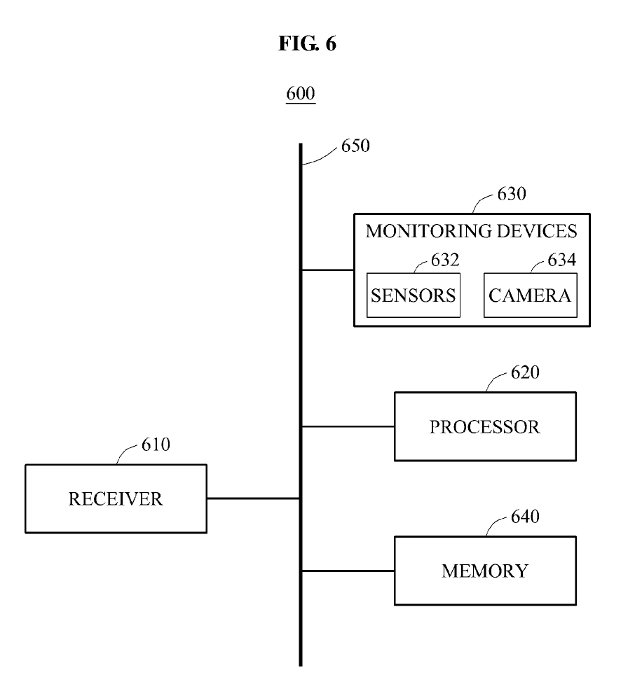

FIG. 6 is a diagram illustrating an example of a driver monitoring apparatus 600. Referring to FIG. 6, the driver monitoring apparatus 600 includes a receiver 610, a processor 620, monitoring devices 630, and a memory 640. The monitoring devices 630 include sensors 632 and a camera 634, for example, an image sensor. The receiver 610, the processor 620, the monitoring devices 630, and the memory 640 may communicate with one another through a bus 650.

The receiver 610 receives a result of detecting a movement of a driver from a wearable device worn on the driver. When a movement of the driver is not detected for a period of time, the wearable device may generate a message indicating no movement of the driver. The wearable device may measure a biosignal of the driver, and determine whether an abnormal health condition occurs in the driver.

The processor 620 controls an activation or inactivation of the monitoring devices 630 provided in a vehicle of the driver based on the result of the detecting a movement of a driver, which is received by the receiver 610. The processor 620 determines whether the driver is in a normal driving state in which the driver is able to drive the vehicle normally using information collected from the monitoring devices 630 activated through by the processor 620.

In response to the message indicating movement of the driver is not present, the processor 620 sets an operation mode of the monitoring devices 630 to be an active mode. In response to not receiving the message indicating an absence of movement of the driver, the processor 620 sets the operation mode of the monitoring devices 630 to be a sleep mode.

The processor 620 estimates whether the driver is drowsy or an abnormal health condition occurs in the driver using the collected information, and determine whether the driver is in the normal driving state based on a result of the estimating.

The monitoring devices 630 monitor a movement of the driver. The monitoring devices 630 include the sensors 632, such as, for example, a biosignal sensor configured to sense a biosignal of the driver, an accelerometer, a gyro sensor, a motion sensor configured to sense a movement of the driver, a tactile sensor configured to sense a contact with the driver, an ultrasonic sensor, and the camera 634 configured to capture an image of the driver.

The processor 620 may also perform at least one method described with reference to FIGS. 2 through 5. The processor 620 may execute a program and control the driver monitoring apparatus 600. A program code to be executed by the processor 620 may be stored in the memory 640. The driver monitoring apparatus 600 may be connected to an external device, for example, a personal computer (PC) and a network, through an input and output device (not shown), and may exchange data.

At least one method described with reference to FIGS. 2 through 5 may be provided in a form of an application operating in a processor in a tablet PC, a smartphone, a wearable device, or in a chip to be embedded in a smartphone, a wearable device, or an intelligent vehicle.

The apparatuses, units, modules, devices, and other components illustrated in FIGS. 1 and 6, which perform the operations described herein with respect to FIGS. 2, 3, 4, and 5 are implemented by hardware components. Examples of hardware components include controllers, sensors, generators, drivers and any other electronic components known to one of ordinary skill in the art. In one example, the hardware components are implemented by one or more processors or computers. A processor or computer is implemented by one or more processing elements, such as an array of logic gates, a controller and an arithmetic logic unit, a digital signal processor, a microcomputer, a programmable logic controller, a field-programmable gate array (FPGA), a programmable logic array, a microprocessor, an application-specific integrated circuit (ASIC), or any other device or combination of devices known to one of ordinary skill in the art that is capable of responding to and executing instructions in a defined manner to achieve a desired result. In one example, a processor or computer includes, or is connected to, one or more memories storing instructions or software that are executed by the processor or computer. Hardware components implemented by a processor or computer execute instructions or software, such as an operating system (OS) and one or more software applications that run on the OS, to perform the operations described herein. The hardware components also access, manipulate, process, create, and store data in response to execution of the instructions or software. For simplicity, the singular term “processor” or “computer” may be used in the description of the examples described herein, but in other examples multiple processors or computers are used, or a processor or computer includes multiple processing elements, or multiple types of processing elements, or both. In one example, a hardware component includes multiple processors, and in another example, a hardware component includes a processor and a controller. A hardware component has any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

The methods illustrated in FIGS. 2-5 that perform the operations described herein with respect to FIGS. 2-5 are performed by a processor or a computer as described above executing instructions or software to perform the operations described herein

Instructions or software to control a processor or computer to implement the hardware components and perform the methods as described above are written as computer programs, code segments, instructions or any combination thereof, for individually or collectively instructing or configuring the processor or computer to operate as a machine or special-purpose computer to perform the operations performed by the hardware components and the methods as described above. In one example, the instructions or software include machine code that is directly executed by the processor or computer, such as machine code produced by a compiler. In another example, the instructions or software include higher-level code that is executed by the processor or computer using an interpreter. Programmers of ordinary skill in the art can readily write the instructions or software based on the block diagrams and the flow charts illustrated in the drawings and the corresponding descriptions in the specification, which disclose algorithms for performing the operations performed by the hardware components and the methods as described above.

The instructions or software to control a processor or computer to implement the hardware components and perform the methods as described above, and any associated data, data files, and data structures, are recorded, stored, or fixed in or on one or more non-transitory computer-readable storage media. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, and any device known to one of ordinary skill in the art that is capable of storing the instructions or software and any associated data, data files, and data structures in a non-transitory manner and providing the instructions or software and any associated data, data files, and data structures to a processor or computer so that the processor or computer can execute the instructions. In one example, the instructions or software and any associated data, data files, and data structures are distributed over network-coupled computer systems so that the instructions and software and any associated data, data files, and data structures are stored, accessed, and executed in a distributed fashion by the processor or computer.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A driver monitoring method, comprising:
determining a movement of a driver based on a wearable device;
controlling an activation of monitoring devices provided in a vehicle based on the determining of the movement of the driver;
collecting information from the monitoring devices, in response to the monitoring device being activated; and
determining whether the driver is able to drive the vehicle using the collected information.

2. The method of claim 1, wherein the wearable device comprises at least one of a biosignal sensor, a motion sensor, an accelerometer, an ultrasonic sensor, a gyro sensor, or an image sensor, and
a movement of the driver is detected by at least one of the motion sensor, the ultrasonic sensor, the accelerometer, the gyrosensor, or the image sensor.

3. The method of claim 1, wherein in response to a movement of the driver not being detected for a period of time, the wearable device is further configured to generate a message indicating no movement of the driver.

4. The method of claim 3, wherein, in response to the message indicating no movement of the driver being generated, the wearable device is further configured to measure a biosignal of the driver and to determine whether an abnormal health condition occurs in the driver.

5. The method of claim 4, wherein, in response to determining an abnormal health condition, the wearable device is further configured to generate a message indicating an occurrence of the abnormal health condition.

6. The method of claim 3, in response to the message indicating no movement of the driver, the method further comprises:
measuring a biosignal of the driver using the wearable device;
and
determining whether an abnormal health condition occurs in the driver based on the measured biosignal.

7. The method of claim 3, wherein the determining of the movement of the driver comprises receiving the message indicating no movement of the driver.

8. The method of claim 3, wherein the controlling of the activation of the monitoring devices comprises setting an operation mode of the monitoring devices to be an active mode in response to receiving the message indicating no movement of the driver.

9. The method of claim 1, wherein the controlling of the activation of the monitoring devices comprises:
setting an operation mode of the monitoring devices to be a sleep mode, in response to determining a movement of the driver.

10. The method of claim 1, wherein the determining of whether the driver is able to drive the vehicle comprises determining whether the driver is able to drive the vehicle using information collected from the monitoring devices and information collected from the wearable device.

11. The method of claim 1, wherein the determining of whether the driver is able to drive the vehicle comprises determining whether the driver is able to drive the vehicle, in response to a switch of a driving mode of the vehicle from an autonomous driving mode to a manual driving mode.

12. The method of claim 1, wherein the determining of whether the driver is able to drive the vehicle comprises:
estimating whether the driver is drowsy or whether an abnormal health condition occurs in the driver using the collected information; and
determining whether the driver is able to drive the vehicle based on the estimating.

13. The method of claim 1, wherein the monitoring devices comprise at least one of a biosignal sensor configured to sense a biosignal of the driver, a motion sensor configured to sense a movement of the driver, a tactile sensor configured to sense a contact with the driver, or a camera configured to capture an image of at least a portion of the driver's body.

14. The method of claim 1, in response to a determination that the driver is not able to drive the vehicle, the method further comprises at least one of:
wirelessly transmitting a warning signal to at least one of a control server and another vehicle traveling around the vehicle; or
transmitting a biosignal of the driver and information about the vehicle to the control server.

15. The method of claim 1, in response to a determination that the driver is not able to drive the vehicle, the method further comprises setting a driving mode of the vehicle to an autonomous driving mode.

16. A non-transitory computer readable medium comprising a program to cause a computer to perform the method of claim 1.

17. A driver monitoring apparatus, comprising:
a receiver configured to receive information, detected from a wearable device, concerning a movement of the driver; and
a processor configured
to control an activation of monitoring devices provided in a vehicle, in response to the information, and
to determine whether the driver is able to drive the vehicle using information collected from the monitoring devices activated, in response to the information indicating no movement of the driver, and
wherein the monitoring devices are configured to monitor a movement of the driver.

18. The apparatus of claim 17, wherein in response to a movement of the driver not being detected for a period of time, the wearable device is further configured to generate a message indicating no movement of the driver, to measure a biosignal of the driver, and to determine, based on the measured biosignal, an occurrence of abnormal health condition in the driver.

19. The apparatus of claim 17, wherein the processor is further configured to set an operation mode of the monitoring devices to be an active mode in response to the information indicating no movement of the driver, and to set the operation mode of the monitoring devices to be a sleep mode in response to the information indicating movement of the driver.

20. The apparatus of claim 17, wherein the processor is further configured to estimate whether the driver is drowsy or whether an abnormal health condition occurs in the driver using the collected information, and to determine whether the driver is in the normal driving state based on the estimating.

21. A driver monitoring method, comprising:
determining a movement of a driver based on at least one of a wearable device or a sensor provided in a vehicle;
controlling an activation of monitoring devices provided in a vehicle based on the movement of the driver;
determining whether the driver is able to drive the vehicle based on information collected from the monitoring devices; and
switching a driving mode of the vehicle to an autonomous driving mode, in response to determining that the driver is unable to drive the vehicle.

22. The method of claim 21, further comprising transmitting a warning signal to another vehicle traveling around the vehicle, in response to determining that the driver is unable to drive the vehicle.

23. The method of claim 21, further comprising transmitting information about the vehicle and a warning signal to a control server, in response to determining that the driver is unable to drive the vehicle.
